(19) United States
(12) Patent Application Publication
(10) Pub. No.: US 2017/0276664 A1
(43) Pub. Date: Sep. 28, 2017

(54) EVAPORATION ON SUPERHYDROPHOBIC SURFACES FOR DETECTION OF ANALYTES IN BODILY FLUIDS

(71) Applicant: The Regents of the University of California, Oakland, CA (US)

(72) Inventors: Michelle Khine, Irvine, CA (US); Jolie McLane, Newport Beach, CA (US); Chun Wu, Rowland Heights, CA (US)

(21) Appl. No.: 15/619,853

(22) Filed: Jun. 12, 2017

Related U.S. Application Data
(62) Division of application No. 14/591,825, filed on Jan. 7, 2015, now abandoned.
(60) Provisional application No. 61/924,359, filed on Jan. 7, 2014.

(51) Int. Cl. G01N 33/493; B01L 3/00; G01N 21/05; G01N 21/25; G01N 33/68; G01N 21/03

(52) U.S. Cl. CPC G01N 33/493; B01L 3/5088; G01N 21/05; G01N 21/251; G01N 33/68; B01L 2300/163; B01L 2300/166; B01L 2400/0406; B01L 2400/088; G01N 2021/035; B01L 3/502792; B01L 2200/12; G01N 2021/0325; B01L 3/502707

(57) ABSTRACT

This disclosure provides a diagnostic system including a detection zone adapted to receive a volume of biological fluid. The detection zone includes a plurality of micro-scale and nano-scale features that render the detection zone superhydrophobic. Analytes (e.g., proteins and/or other molecules) are concentrated when the volume of biological fluid is allowed to evaporate on the detection zone. Concentrating the analytes in the detection zone by evaporation can advantageously increase the sensitivity of detection of the analyte. In various implementations, microfluidic channels can be integrated with the diagnostic system to convey the volume of biological fluid to the detection zone. In various implementations, the microfluidic channels can have a lower hydrophobic characteristic than the surrounding to realize self-driven microfluidic channels that convey the biological fluid to the detection zone without using any external devices.

### STATEMENT REGARDING FEDERALLY SPONSORED R&D

The inventions were made with government support under N66001-4003 awarded by Defense Advanced Research Projects Agency (DARPA), 442870-19900 awarded by the Undergraduate Research Opportunities Program, DGE 0549479 awarded by the National Science Foundation (Lifechips), 442870-30031 awarded by the National Institute for Health New Innovator Program.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

Field of the Invention

This application pertains to microfluidic devices, including substrates for handling and concentrating fluid samples in connection with diagnostic apparatuses.

Description of the Related Art

Microfluidics is a field that has been widely explored. Generally, traditional microfluidics are created using photolithography and require molding, bonding, punching, tubing, pumping, valving, and external pressure. These systems are very complex and can be subject to blockage of the small microfluidic channels.

Solid matter in a solution or suspension can be analyzed by permitting evaporation to reduce or eliminate the volume of liquid. On a glass slide or other general surface, the solid matter that remains after evaporation may be too diffuse for accurate measurement or may spread much more widely than needed for analysis.

While it is known to create superhydrophobic (SH) on a surface, standard techniques for doing so involve a chemical modification of the surface. Such chemical modifications are not compatible with certain biological applications.

### SUMMARY OF THE INVENTION

Superhydrophobic (SH) surfaces are used for many applications because of their unique behavior. Water beads up on a SH surface, has a weak adhesion to the SH surface, and slides rather than adheres to the surfaces. When a water droplet evaporates on a SH surface, the weak adhesion allows the footprint of the droplet to continually shrink until the fluid completely evaporates. During evaporation, molecules are concentrated and confined to a smaller final footprint, thus enhancing the concentration of molecules compared a flat surface. Further, the proposed SH surfaces can be used to create self-driven microfluidic devices. When the SH surface is patterned with superhydrophilic regions, fluid will only wet the superhydrophilic regions and not wet the SH regions. Thus, flow can be driven by a high contrast in wettability rather than an external source. Because these channels are self-driven, open-channels can be created, negating the need for external equipment. In addition, the proposed SH surfaces are also phobic to bodily fluids such as blood, saliva, and urine, and these fluids can be used as a replacement for water on the SH surfaces.

The SH surfaces are created using a structural modification, and a fluid droplet sits on the peaks of the structurally modified surface with minimal adhesion. During evaporation, liquid evaporates into the atmosphere at the air-liquid interface of the droplet, and the surface tension locally increases at the surface of the water droplet. This increase in surface tension is great enough to depin the fluid droplet from the SH surface and to pull the footprint (contact area where the droplet attaches to the SH surface) of the droplet inward. In this context, “depin” means that the water droplet's contact line (outer circumference where the droplet attaches to the surface) detaches from the surface because the droplet's surface tension is greater than the adhesive force of the SH surface. When the molecules in the water relax and tension is balanced due to depinning, the droplet repins to another peak of the SH surface. In this context, “repin” means that the weak adhesion of the SH surface is a strong enough to reattach to the water droplet's contact line and hold the droplet in its ball-like shape. This depinning and repining continues until the surface tension is not stronger than the pinning force. The fluid eventually completely evaporates in the atmosphere, and only the dry contents of the droplet are left on the surface. Thus, molecules in the droplet are concentrated, and due to the decrease in footprint size, the concentration effect is greater on a SH surface compared to a flat surface.

Superhydrophilic regions are selectively patterned on the surface using a chemical modification. A SH substrate is created, and a negative mask is used to cover the SH regions during chemical treatment. The mask can be created using polyolefin tape, but is not limited to this method. Superhydrophilicity can be achieved by plasma or corona treatment to deposit hydrophilic oxygen molecules on the surface. Silica can also be deposited on the surface as a hydrophilic agent. Changing the surface to hydrophilic is not limited to plasma, corona, or silica, but rather, can be created using many hydrophilic agents. When fluid contacts the patterned substrate, fluid will not wet the SH regions and will only wet the superhydrophilic regions. Superhydrophilic channels can be created, and fluid will flow along the channels without an external source. Fluid flows in the channels due to internal droplet pressure as well as the high affinity to the superhydrophilic surface. These self-driven, open-channel microfluidic devices differ from traditional microfluidics because they negate one or more of external pumping equipment, tubing, and/or valving. Self-driven devices also advantageously are not subject to clogging as are closed channels, and are less prone to nonspecific protein adsorption from walls of channels. They are also compatible with small volumes of fluids, yield rapid results, and can used as or in a portable device.

Bodily fluid are also compatible with the SH surfaces, and blood, saliva, and urine can be used as the testing fluid on the SH surfaces. SH surfaces have also been shown to prevent blood clotting, and the proposed surfaces can be used as an anticoagulation surface.

In one application, a diagnostic system is provided that includes a platform and a detector. The platform has an exposed surface, at least a portion of which comprises a high hydrophobic (e.g., superhydrophobic) characteristic. The detector is configured to be directed toward the surface. The detector and/or the system detect a property of a sample disposed on the surface. The system enables the detector to detect one or more analytes in low concentration in a fluid.

Of course, the system can also detect one or more anlaytes in higher concentration. But, unlike other systems low concentrations can be detected by the system. One embodiment encompasses systems that can detect BSA in concentrations as low as 5 μg/mL.

In another application, a point-of-care device is provided that includes a detector and a platform. The platform has an open expanse of solid low cost plastic. The expanse includes an exposed boundary portion comprising a superhydrophobic surface. The boundary portion at least partially surrounds a channel that has hydrophobicity less than that of the boundary portion. The channel can be superhydrophilic in some embodiments. The difference in hydrophobicity and/or hydrophilicity preferably is sufficient to drive a sample along the channel.

One innovative aspect of the embodiments described herein can be implemented in a diagnostic system, comprising a platform and a detector. The platform includes an exposed surface, at least a portion of which comprises a detection zone having a high hydrophobic characteristic. The detector is configured to be directed toward the surface and to detect a property of a sample of a fluid disposed on the detection zone of the surface. The system can advantageously allows the detector to detect one or more analytes in the fluid sample.

Another innovative aspect of the embodiments described herein can be implemented in a point-of-care device, comprising a polymer platform and a detector. The platform includes an open expanse including at least one microfluidic channel surrounded by a region having a hydrophobic characteristic greater than a hydrophobic characteristic of the channel, the expanse including a detection zone in fluidic communication with the channel. The detector is configured to be aligned with the detection zone.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments.

FIGS. 1A-1D illustrate evaporation on a SH surface. FIG. 1A illustrates that fluid evaporates from the droplet into the atmosphere at the air-liquid interface. FIG. 1B illustrates that particles are concentrated during evaporation. FIG. 1C illustrates a 2 μL droplet with food dye shrinking during evaporation, and FIG. 1D molecules concentrated after evaporation. Scale bars are 500 μm in length.

FIG. 2 illustrates an implementation of a method to fabricate a biocompatible superhydrophobic (SH) surface.

FIGS. 3A and 3B illustrate images of a SH surface under different magnifications.

FIGS. 4A-4C illustrate different parameters of a fluid droplet for different volumes of the droplet. FIG. 4A shows that as the droplet's volume increases, the diameter, height, and contact length of the droplet increase. FIG. 4B illustrates that the ratio of height to diameter (H/D) decreases as volumes increases due to gravity, and calculated volume compares well with deposited volume. FIG. 4C illustrates that internal droplet pressure increases as the volume decreases, and the CA consistently stays above 150° for all volumes.

FIG. 5 includes images of droplet evaporation. The diameter, height, contact length, and volume decrease, but the CA remains SH. Eventually, the droplet completely evaporates.

FIGS. 6A-6F characterizes different parameters of a droplet of water as it evaporates on a SH surface. FIG. 6A illustrates the variation in the diameter with time for different volumes of water droplet. FIG. 6B illustrates the variation in the height with time for different volumes of water droplet. FIG. 6C illustrates the variation in the contact length with time for different volumes of water droplet. FIG. 6D illustrates the variation in the calculated volume with time for different volumes of water droplet. FIG. 6E illustrates the variation in the pressure with time for different volumes of water droplet. FIG. 6F illustrates the variation in the contact angle with time for different volumes of water droplet.

FIGS. 7A-7F compares the different parameters of 2 μl droplet of food dye with concentrations from 0.001%, 0.01%, 0.1% and 1% with a 2 μl droplet of water as they evaporate on a SH surface. FIG. 7A illustrates the variation in the diameter with time as the different droplets evaporate. FIG. 7B illustrates the variation in the height with time as the different droplets evaporate. FIG. 7C illustrates the variation in the contact length with time as the different droplets evaporate. FIG. 7D illustrates the variation in the calculated volume with time as the different droplets evaporate. FIG. 7E illustrates the variation in the pressure with time as the different droplets evaporate. FIG. 7F illustrates the variation in the contact angle with time as the different droplets evaporate.

FIG. 8A-1 is an image of droplets of food dye having different concentrations 0.0%, 0.001%, 0.1% and 1% that are placed on a SH surface at an initial time. FIG. 8A-2 is an image of the droplets at a later time as the droplets evaporate over time. FIG. 8A-3 shows the colorimetric signal intensity as a function of time as the droplets of food dye evaporate from a SH surface.

FIG. 8B-1 and 8B-2 shows images of droplets of food dye having different concentrations 0.001%, 0.01%, 0.1% and 1% placed on a flat surface that is devoid of micro-scale and/or nano-scale features and thus non SH. FIG. 8B-3 shows the colorimetric signal intensity as a function of time as the droplets of food dye evaporate from a non-SH surface.

FIGS. 9A-9F compares the different parameters of 2 μl droplet of food dye with concentrations of 0.01% and 0.1%, 2 μl droplet of 5 μgm/ml bovine serum albumin (BSA) solution, 2 μl droplet of 25 μ/ml BSA solution with a 2 μl droplet of water as they evaporate on a SH surface. FIG. 9A illustrates the variation in the diameter with time as the different droplets evaporate. FIG. 9B illustrates the variation in the height with time as the different droplets evaporate. FIG. 9C illustrates the variation in the contact length with time as the different droplets evaporate. FIG. 9D illustrates the variation in the calculated volume with time as the different droplets evaporate. FIG. 9E illustrates the variation in the pressure with time as the different droplets evaporate. FIG. 9F illustrates the variation in the contact angle with time as the different droplets evaporate.

FIG. 10A-1 shows the increase in the colorimetric signal intensity over time of the detection dye resulting from evaporation on a SH surface. FIG. 10A-2 illustrates the image of droplets of detection dye disposed on a SH surface mixed with different concentrations of BSA ranging from 0 μg/ml to 800 mg/ml. FIG. 10A-3 shows the colorimetric signal intensity for different concentrations of BSA when mixed with detection dye disposed on a SH surface. FIG. 10B-1 illustrates the image of droplets of detection dye disposed on a flat surface (or non-SH surface) mixed with different concentrations of BSA ranging from 0 μg/ml to 800 mg/ml. FIG. 10B-2 shows the colorimetric signal intensity for different concentrations of BSA when mixed with detection dye disposed on a non-SH surface.

FIGS. 11A and 11B show different implementations of a hydrophilic microfluidic channel surrounded by a hydrophobic region.

FIGS. 12A and 12B show different implementations of methods to fabricate hydrophilic microfluidic channels on a hydrophobic surface.

### DETAILED DESCRIPTION OF THE PREFERRED EMBODIMENT

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

This application describes inventions that exploit hydrophobicity, hydrophilicity, and or gradients of these properties. For example, the behavior of water on a superhydrophobic (SH) surface can be leveraged to concentrate biomolecules for enhanced detection of proteins. As the volume decreases during evaporation, the contact area also decreased due to the weak adhesion of water to a SH surface, yielding small volumes that are unachievable on a smooth, flat surface. During evaporation, the volume is reduced to a tiny fraction of the pre-evaporation volume, e.g., up to 402×, and the contact area also is greatly reduced, e.g., up to 4.75×. By decreasing the volume, the concentration of a solution with few particles increases, and thus, a SH surface achieves more concentrated solutions compared to flat surfaces. These higher concentrations are easier to detect and can be detected with less costly techniques (e.g., by eye in a colorimetric assay). Evaporation on these surfaces is compatible with protein solutions, and in a colorimetric assay, the signal is enhanced. With the SH surface, 5 μg/mL of protein can be detected, a 10-fold improvement compared to flat surfaces.

In certain embodiments, a platform and/or a system is provided for detection of proteins in body fluids such as urine. These embodiments can be used advantageously to diagnose or monitor patients with various conditions. One condition that can be monitored or diagnosed with this method is pre-eclampsia during pregnancy. Systems and devices that can be used to monitor or diagnose with pre-eclampsia during pregnancy are discussed below.

With the low-cost fabrication method and simple technique, highly sensitive detection can be achieved in a low-cost platform.

### Superhydrophobic (SH) Surfaces

A surface is considered superhydrophobic (SH) when water prefers to bead up and roll off the surface rather than wet the surface. More specifically, a SH surface has a water contact angle (CA) greater than 150° and a sliding angle (SA) less than 10°. This unique behavior of water is caused by the high surface tension of water, the low surface energy of the substrate, and the minimal adhesion between water and the surface. The low surface energy and minimal adhesion can be attributed to multiscale features, ranging from micro to nano. This hierarchy of features traps air pockets between the surface and water, and the water droplet only contacts the peaks of the multiscale structures. Therefore, the multiscale features are key to achieve superhydrophobicity.

When air is trapped between water and the surface, the surface is in the Cassie-Baxter regime, and the water droplet has poor adhesion with the surface. Without any loss of generality, when water is in direct contact with the surface, such as for example, when there are no air pockets between water and the surface, the surface is in the Wenzel regime, and the water droplet has good adhesion with the surface. A SH surface in the Cassie-Baxter regime can transition to the Wenzel regime when the balance of forces is disrupted. Applying pressure can disrupt this balance and change a water droplet from balancing on the peaks to sinking into the multiscale structures. A water droplet can naturally transition from Cassie to Wenzel due to a change in internal droplet pressure as the droplet's size decreases. Internal droplet pressure is inversely related to the droplet size and can be quantified by ΔP=2γ/R, where γ is the surface tension of the fluid, and R is the radius of the droplet. Thus, smaller volumes apply larger pressures at the surface and are capable of overcoming the energy required to transition from the Cassie to Wenzel regime.

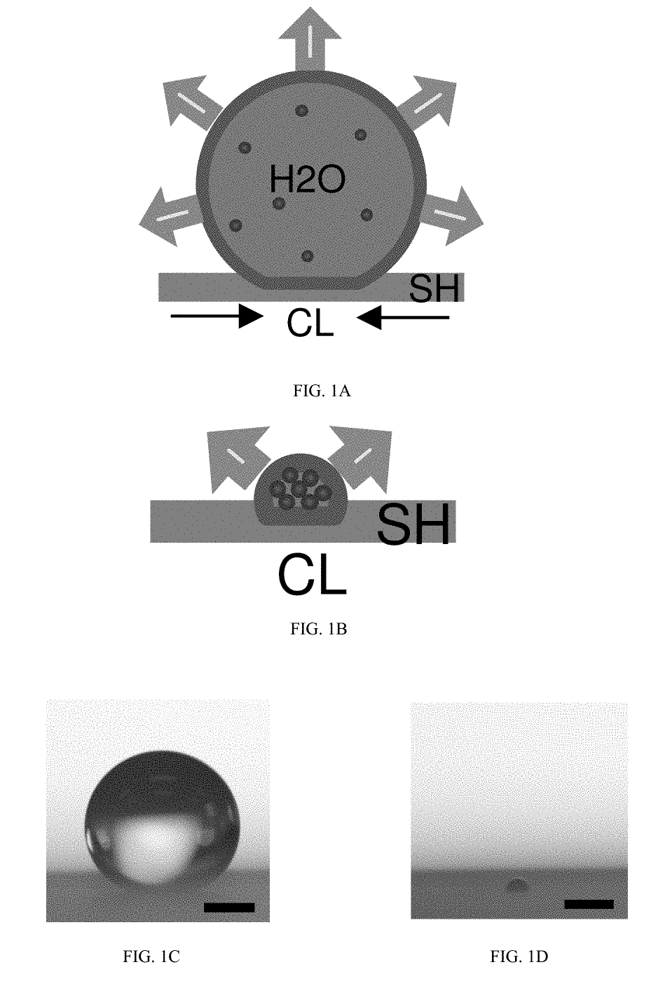

Small volumes can be achieved when fluid evaporates from a droplet. When a droplet of fluid evaporates into the atmosphere, the balance of forces at the air-liquid interface is constantly changing, and the droplet's surface tension is constantly applying an inward force. On a flat surface, the adhesion of water to the surface is great enough to keep the contact line (air-liquid-solid interface) pinned to the surface, and the droplet's contact area remains constant. Due to convective forces at the contact line, molecules in the droplet are pulled toward the contact line, and the molecules evaporate into a coffee ring pattern, making inconsistent patterns of solution. On a superhydrophobic surface, however, there is poor adhesion between the solid and the liquid, and the increased surface tension from evaporation is greater than the pinning force, preventing the water droplet from staying pinned to its initial contact area. Rather, the droplet's contact line slides freely across the SH surface during evaporation, and the contact area of the fluid continually decreases, concentrating molecules within a confined contact area, as shown in FIGS. 1A-1D. Therefore, as volume decreases during evaporation on a SH surface, contact area also decreases due to pinning and depinning of water. This pinning and depinning ends when the droplet's internal Laplace pressure finally overcomes the forces from the air pockets. The droplet then transitions from Cassie to Wenzel, and fluid will stay pinned at this reduced contact area until the fluid is fully evaporated. Because the contact area decreases on a SH surface, the volume is further decreased on a SH surface compared to a flat surface.

Further, as volume decreases, concentration increases and thus, the concentrating effect is enhanced on a SH surface compared to a flat surface because of the decreased volume in a smaller footprint. Thus, diagnostic systems and methods employing evaporating droplets on SH surfaces can be used to increase sensitivity in detection of proteins and other molecules present in biological fluids. Furthermore, it would be advantageously to fabricate such diagnostic systems inexpensively.

Systems and methods discussed herein employ evaporating fluids on SH substrates to enhance detection of protein in assays (e.g., colorimetric assays). The SH substrates can be manufactured simply thereby realizing manufacturing cost reduction. Dyes and proteins incorporated in the fluid are concentrated when the fluid evaporates on the SH substrates. Accordingly, the systems and methods described herein can be used to concentrate biological solutions to increase detection sensitivity for biological testing on a low-cost platform.

### Fabrication of Superhydrophobic (SH) Surfaces

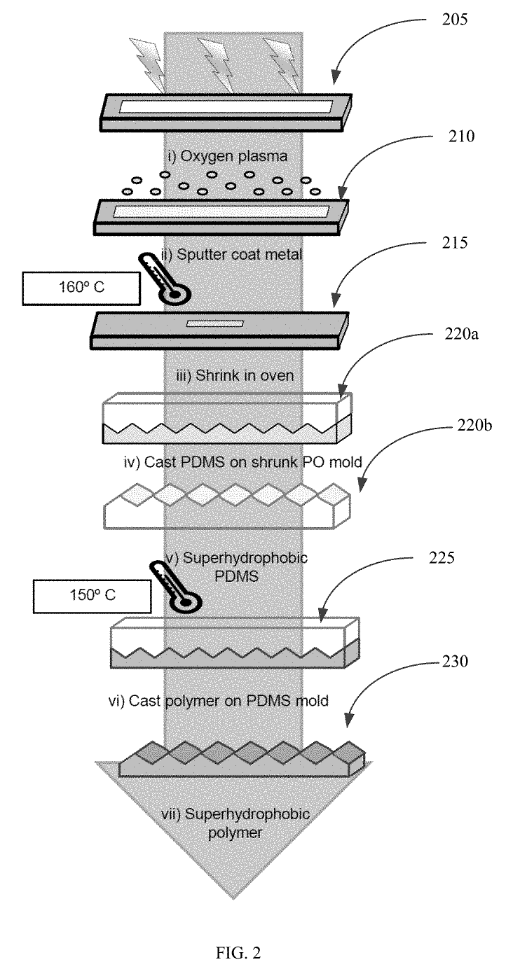

FIG. 2 illustrates an implementation of a method of manufacturing SH surfaces. The method comprises depositing a layer of metal on a polymer material as illustrated in block 210. The polymer material can include a layer of prestressed thermoplastic polyolefin (PO) or a shrink film PO. The layer of metal can be deposited by sputter coating the polymer material. In various implementations, the metal layer can have a thickness between about 5 nm and about 35 nm. The metal layer can comprise Silver, Gold, Calcium or any other suitable metal. The metal layer can be stiff in some implementations. In some implementations, the polymer material can be plasma treated such as, for example, oxygen plasma treated prior to depositing the metal layer as shown in block 205. The polymer material with the metal layer is heated to a temperature between about 150 degree Celsius and about 200 degree Celsius (e.g., at a temperature of about 160 degree Celsius), as shown in block 215. The polymer material can be heated by being placed in an oven or on a hot plate. When heated the polymer material shrinks such that the metal layer deposited on the polymer material buckles and folds resulting in micro- to nanoscale features on the surface. In various implementations, the size of the polymer material can shrink by as much as about 95% of its original size. The metal coated shrunk polymer material is molded onto polydimethylsiloxane (PDMS) (e.g., McMaster Carr PDMS) and/or other moldable polymers as shown in block 220a to realize a PDMS mold with features that are inverse of the micro- to nanoscale features on the metal surface as shown in block 220b. The PDMS mold can be molded or cast with another polymer as shown in block 225 to achieve a SH surface as shown in block 230. In various implementations, PDMS mold can be hot embossed to polyethylene (PE) to realize a SH polyethylene (PE) surface. In an embodiment of a SH polyethylene surface, the contact angle (CA) of a water drop was about 154.6 degrees and the sliding angle (SA) was about 5.6 degrees. Without any loss of generality, the contact angle of water drop on the SH surface fabricated by this method can be greater than 150 degrees and the sliding angle can be less than 25 degrees.

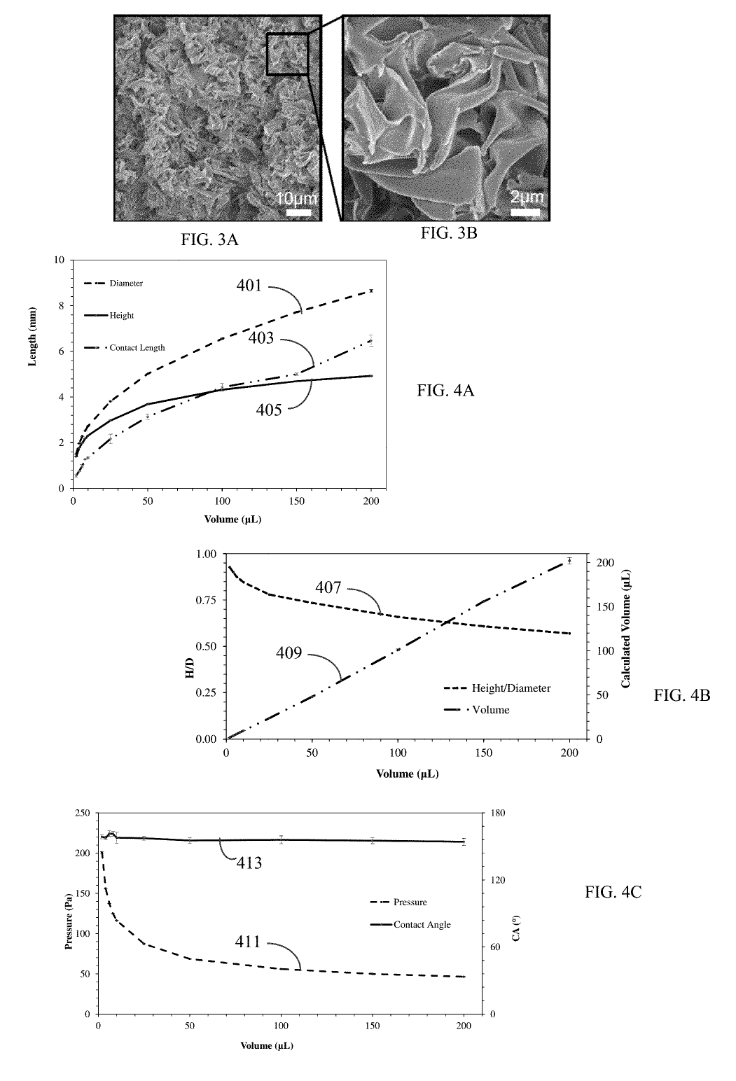

FIG. 3A is an image of the metal surface when the polymer is shrunk. As observed from FIG. 3A micro-scale and nano-scale features develop on the metal surface, when the polymer is heat shrunk. The scale bar in FIG. 3A has a length of 10 μm. FIG. 3B illustrates a portion of the surface illustrated in FIG. 3A when observed under higher magnification. The scale bar in FIG. 3B has a length of 2 μm.

### Evaporation of Drops on a SH Surface and the Resulting Concentrating Effect

To understand the evaporation of fluids on a SH surface and the concentrating effect of proteins and dyes incorporated in the fluid due to evaporation the tests described below were performed. For the purpose of testing, purified deionized (DI) water was used to characterize evaporation on the SH surfaces. Food dye (Market Pantry) was tested to quantify signal enhancement. Protein colorimetric detection dye (Biorad) was also used to quantify signal enhancement. In various implementations, the protein colorimetric detection dye was filtered and diluted with DI water before testing. Bovine serum albumin (BSA) (Biorad) was the protein solution tested to show enhanced detection of biological fluids. Food dye and BSA were also diluted with DI water for testing purpose.

To understand the behavior of water droplets on a SH surface, drops of water having different volumes between 1 μl-200 μl were deposited on the SH surface. The diameter (D), height (H), and contact length (CL) dimensions, Laplace pressure, and CA of water droplets were characterized. In addition, the deposited droplet volume was compared to the calculated volume using software. The height/diameter (H/D) was also calculated. Droplet diameter, height, and contact length were quantified by comparing a known reference dimension to the droplet dimensions. Internal droplet Laplace pressure was calculated with the measured droplet radius and the surface tension of water. Volume and CA measurements were analyzed using the low-bond axisymmetric drop shape analysis (LB-ADSA) software in ImageJ.

FIG. 4A, shows droplet dimensions diameter (represented by curve 401), height (represented by curve 403) and contact length (CL) (represented by curve 405) for droplets with different volumes ranging from 1 μl-200 μl. FIG. 4B illustrates the height to diameter ratio (H/D) ratio (represented by curve 407) and the calculated volume versus the volume of the droplet deposited on the SH surface (represented by curve 409). FIG. 4C illustrates the variation of Laplace pressure (Pa) (represented by curve 411) and CA with respect to droplet volume (represented by curve 413). It is noted from FIG. 4A that as expected the droplet dimensions increase as droplet volume increases. The diameter and CL continually increase, but the height rate of increase is less for larger volumes due to the large mass and gravity pulling the droplet toward the surface. From FIG. 4B, it is noted that the H/D ratio is less than 1 indicating that height is consistently less than diameter in part due to gravity. It is further noted that the H/D ratio decreases as the volume of the droplet increases indicating that droplets with smaller volumes have a more spherical shape than droplets with larger volumes. It is also noted that the calculated volume is consistent with the applied volume.

It is noted from FIG. 4C that water droplets with a volume greater than 50 μL have relatively low and constant Laplace pressures ranging from 46-69 Pa for 50-200 μL. As the volume decreases though, the internal droplet pressure increases, and a 24 μL droplet has a pressure greater than 200 Pa, showing that the SH surfaces can withstand high pressures. CA remains above 150° for all volumes, indicating that superhydrophobicity is unaffected by the droplet volume and that the balance of forces at the contact line maintains superhydrophobicity.

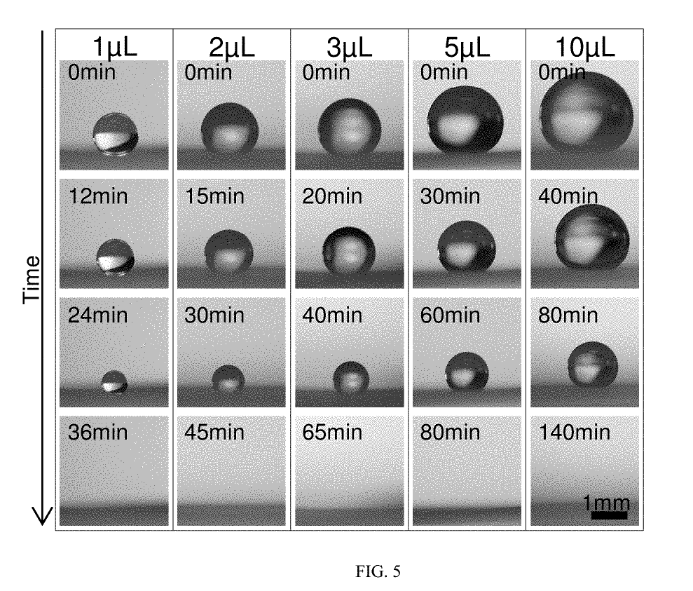

For the purpose of testing evaporation of fluids on a SH surface, droplets of water having volumes ranging from 1-10 μL were deposited on the SH surface. FIG. 5 shows images of water droplets with volumes of 1 μl, 2 μl, 3 μl, 5 μl and 10 μl at different times. The images were taken with a Cannon EOS Rebel camera and macro lenses.

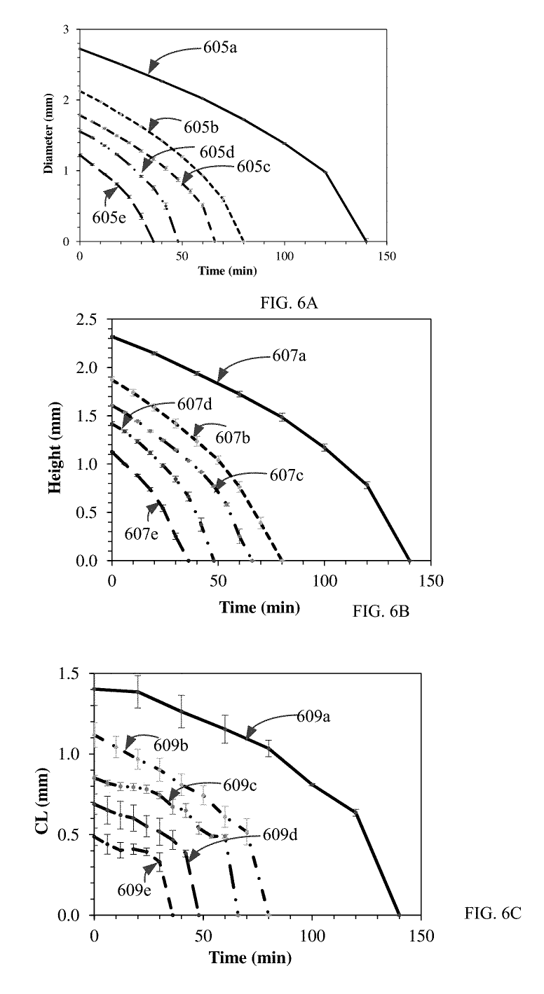

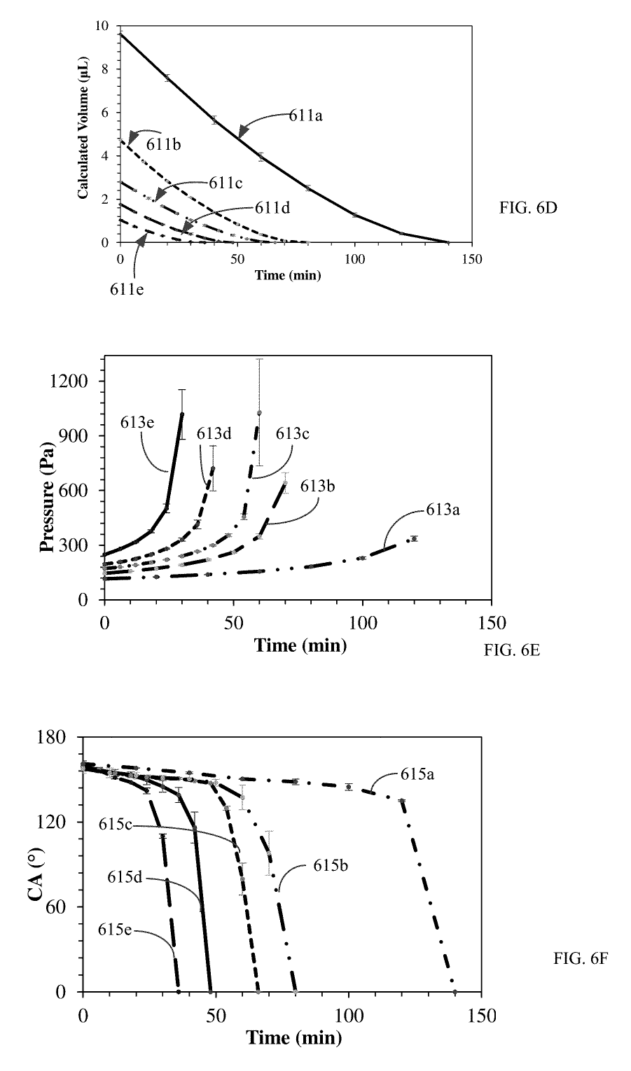

FIGS. 6A-6F show the variation in diameter, height, contact length (CL), calculated volume, pressure and contact angle over time for the water droplets with volumes of 1 μl, 2 μl, 3 μl, 5 μl and 10 μl. With reference to FIGS. 6A-6F, curves 605a, 607a, 609a, 611a, 613a and 615a represent the variation in diameter, height, contact length (CL), calculated volume, pressure and contact angle over time respectively for a water droplet with a volume of 10 μl. Curves 605b, 607b, 609b, 611b, 613b and 615b represent the variation in diameter, height, contact length (CL), calculated volume, pressure and contact angle over time respectively for a water droplet with a volume of 5 μl. Curves 605c, 607c, 609c, 611c, 613c and 615c represent the variation in diameter, height, contact length (CL), calculated volume, pressure and contact angle over time respectively for a water droplet with a volume of 3 μl. Curves 605d, 607d, 609d, 611d, 613d and 615d represent the variation in diameter, height, contact length (CL), calculated volume, pressure and contact angle over time respectively for a water droplet with a volume of 3 μl. Curves 605b, 607b, 609b, 611b, 613b and 615b represent the variation in diameter, height, contact length (CL), calculated volume, pressure and contact angle over time respectively for a water droplet with a volume of 2 μl. Curves 605e, 607e, 609e, 611e, 613e and 615e represent the variation in diameter, height, contact length (CL), calculated volume, pressure and contact angle over time respectively for a water droplet with a volume of 1 μl.

As noted from FIG. 5, size of the water droplets decrease over time due to evaporation. Larger volumes take longer to evaporate (about 30 min for water droplet with 1 μL volume compared to about 140 min for water droplet with 10 μL volume). It is noted from FIGS. 6A-6D that droplet dimensions such as diameter, height, contact length, and calculated volume also decrease as a function of time. Indicative of a SH surface, the contact length decreases during evaporation because the surface is in the SH Cassie regime, and thus the pinning and depinning phenomenon occurs.

All volumes maintain a SH Cassie state during the initial evaporation. The volume at which the CA falls below SH values (i.e. transitions from Cassie to Wenzel) is approximately 300 nL, and the corresponding transition pressure is approximately 360 Pa. This internal pressure overcomes the force from air trapped beneath the water droplet and allows the fluid to collapse into the multiscale features (i.e. pin to the surface) indicating that the substrates disclosed herein can withstand high pressures before transitioning. Eventually, all water evaporates into the atmosphere, and no footprint remains after evaporation of pure water. Multiple evaporation studies of water were performed on the same substrate (until fluid was fully evaporated), and all data yielded SH characteristics, showing that the transition from the Cassie to the Wenzel regime is reversible once air pockets are introduced again.

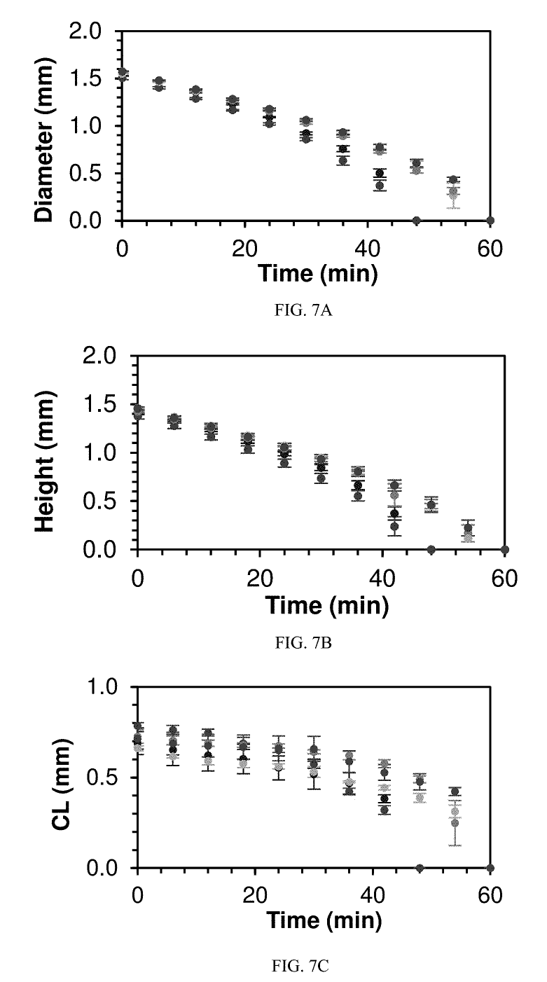

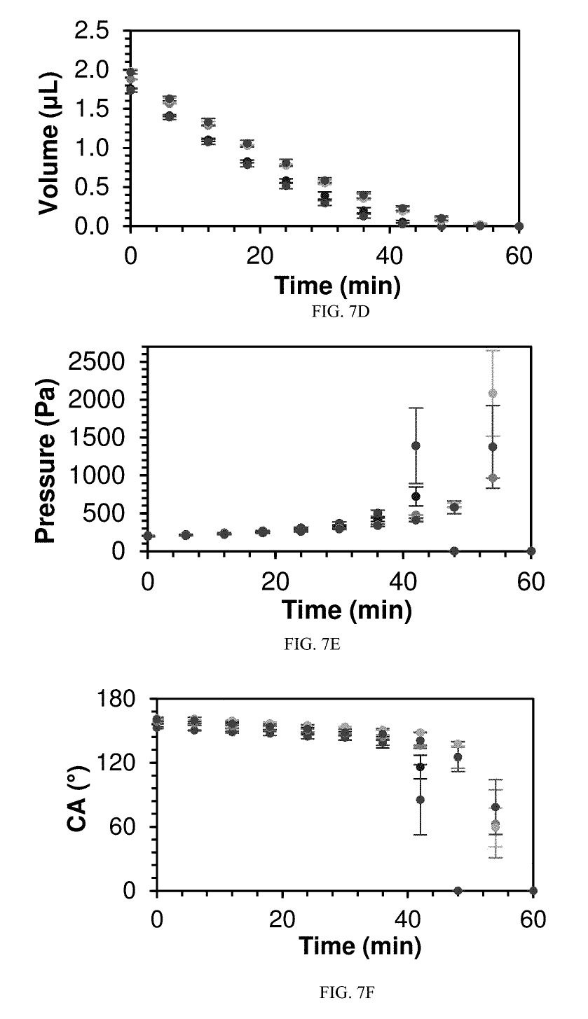

Different solutions of food dye and/or BSA in water were also evaporated on the SH surfaces. To understand the differences between evaporation of water and evaporation of different solutions of food dye and/or BSA in water various parameters of 2 droplets of the different solutions were measured over a time interval and compared to the dimensions of 2 μL droplet of water over the same time interval. The parameters such as, for example, diameter, height, contact length, volume, pressure, and CA were obtained from images of the droplets of the different solutions taken every 6-20 minutes until solutions were completely evaporated. All measurements were taken at room temperature with ambient conditions. FIGS. 7A-7F compares the different parameters of 2 μl droplet of food dye with concentrations from 0.001%, 0.01%, 0.1% and 1% with a 2 μl droplet of water as they evaporate on a SH surface. Low concentrations of food dye had similar evaporation rates and dimensions as that of pure water, which could be attributed to the low presence of molecules. High concentrations of food dye had slower evaporation rates as well as higher diameters and contact lengths compared to pure water. The presence of molecules in the solution blocks interactions at the air-liquid interface to slow down evaporation and increases interactions with the surface, thus increasing the contact length. CA values remained consistent with SH properties for all food dye concentrations until volumes were small enough to pin the droplet to the surface (approximately 300-500 nL and approximately 300-350 Pa, similar to pure water). Solutions with higher concentrations left visible particle residue in the footprint after all fluid was evaporated.

Initially, the calculated volume of food dye is 1.89±0.11 μL, and the volume decreases to 17±9 nL before evaporation is complete. Therefore, particles in the droplet are concentrated on average at least 111× with a maximum of 402× in the measured data. Note that after evaporation, solutions will result in a dry pellet, and volume measurements are based on the time point before evaporation is complete. Therefore, the volume reduction is even lower than calculated values, and thus the concentration enhancement could be greater than predicted. In addition, the contact area decreases from 0.41±0.06 mm2 to 0.09±0.04 mm2, which is a 4.75× reduction in contact area due to evaporation. Therefore, particles in the droplet are highly concentrated due to evaporation on a SH surface.

Since all droplets remained in the SH Cassie state until extremely low volumes and high pressure were reached, volumes of water larger than 10 μL were not characterized. Without any loss of generality, larger volumes will follow the same trend and remain SH until their internal pressure becomes great.

The concentration effect resulting from evaporation on a SH surface was measured by colorimetric methods using Food dye and detection dye. To measure the concentration effect colorimetrically, images were taken from a top-down view. Lighting was controlled by a dark box and a single light source, and images were taken in series every 10 minutes until solutions were completely evaporated.

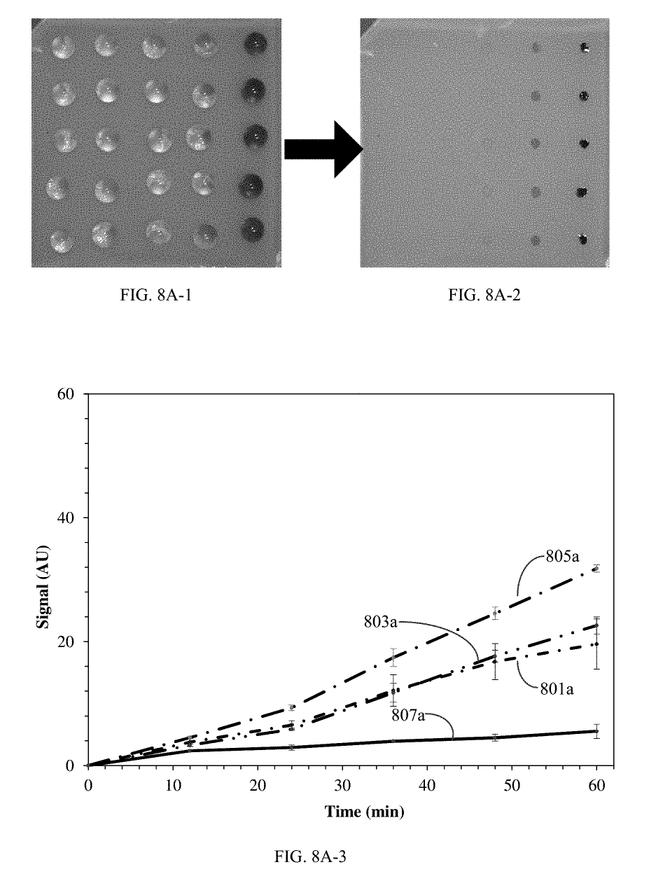

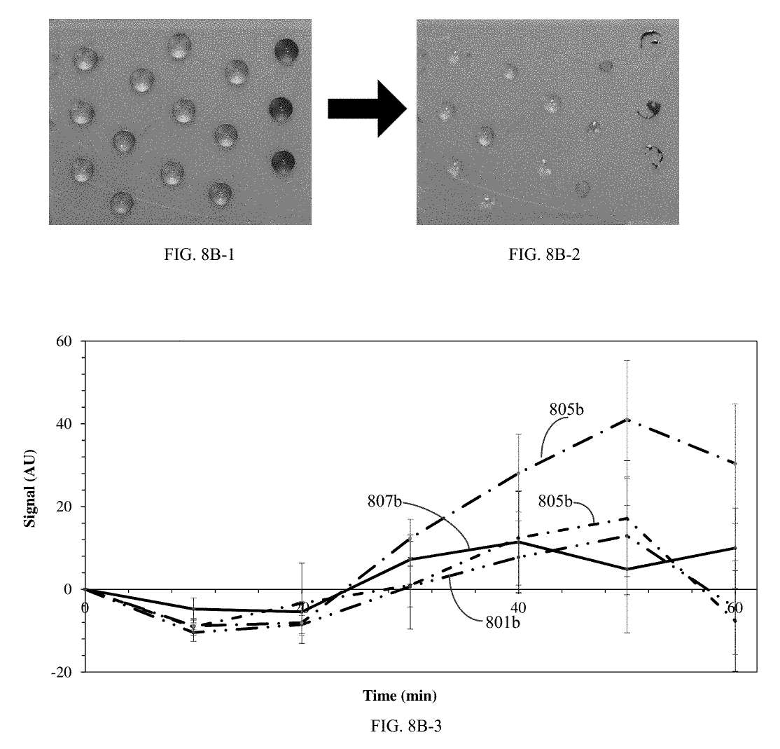

FIG. 8A-1 is an image of droplets of food dye having different concentrations 0.001%, 0.01%, 0.1% and 1% that are placed on a SH surface at an initial time. FIG. 8A-2 is an image of the droplets at a later time as the droplets evaporate over time. FIG. 8A-3 shows the colorimetric signal intensity as a function of time as the droplets of food dye evaporate from a SH surface. Colorimetric signal intensity was measured as color intensity in Adobe Photoshop. From FIGS. 8A-1 and 8A-2 it is observed that droplets with high concentrations of food dye left powder residue after evaporation. It was also visually observed that the color intensities saturated as the concentration of food dye increased. Furthermore, as droplets of food dye evaporate, the colorimetric signal increases as shown in FIGS. 8A-3. Curve 801a represents the colorimetric signal over time for a 0.001% solution of food dye; curve 803a represents the colorimetric signal over time for a 0.01% solution of food dye; curve 805a represents the colorimetric signal over time for a 0.1% solution of food dye; and curve 807a represents the colorimetric signal over time for a 1% solution of food dye. Low concentrations of food dye become more concentrated and the signal intensity continually increases until the droplet is fully evaporated at 60 min. Signal increase is dependent on the initial food dye concentration, and solutions with more particles initially have the greatest signal enhancement. However, the 1% food dye initially has a high colorimetric signal, as illustrated in FIG. 8A-3, and therefore, the reduced signal increase is due to signal saturation.

FIG. 8B-1 and 8B-2 shows images of droplets of food dye having different concentrations placed on a flat surface that is devoid of micro-scale and/or nano-scale features and thus non SH. FIG. 8B-3 shows the colorimetric signal intensity as a function of time as the droplets of food dye evaporate from a non-SH surface. Curve 801b represents the colorimetric signal over time for a 0.001% solution of food dye; curve 803b represents the colorimetric signal over time for a 0.01% solution of food dye; curve 805b represents the colorimetric signal over time for a 0.1% solution of food dye; and curve 807b represents the colorimetric signal over time for a 1% solution of food dye. A comparison of FIGS. 8A-3 and 8B-3 indicates that the colorimetric signal enhancement is greater and more consistent on a SH surface than on a flat surface (or non-SH surface). This can be attributed at least in part to the particles not being confined to a specific contact area.

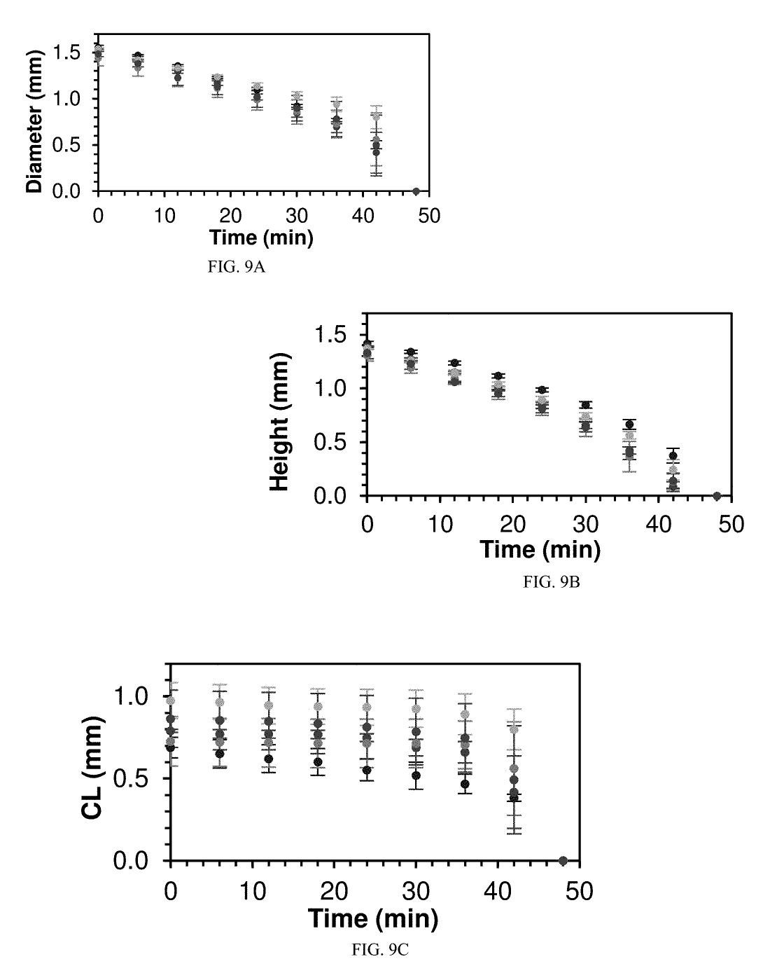

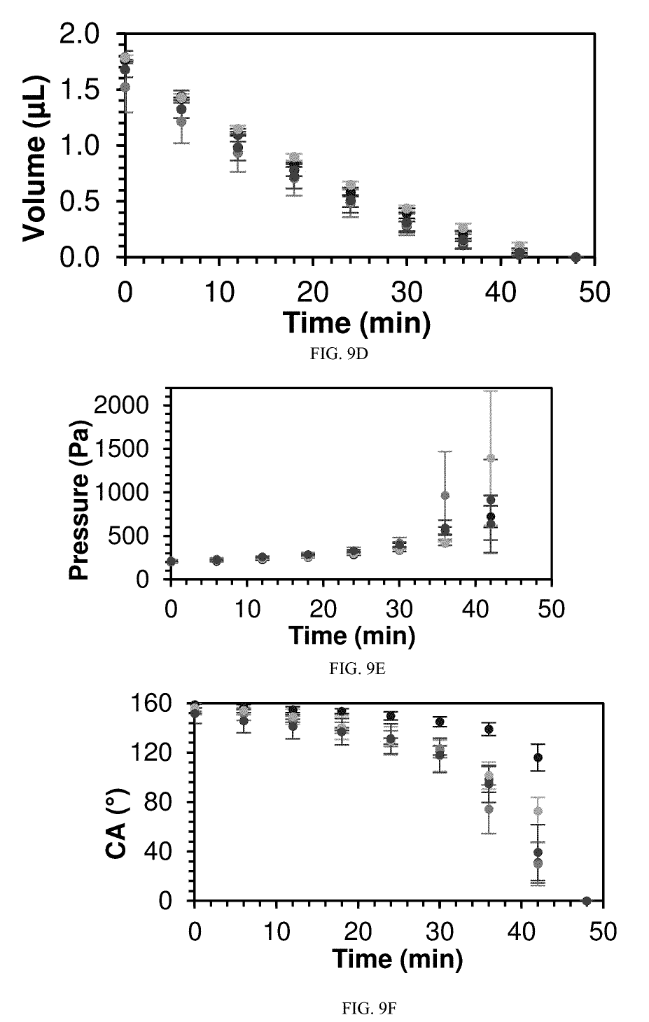

FIGS. 9A-9F illustrate the variation of various parameters such as diameter, height, contact length, calculated volume, pressure and contact angle for a 2 μl droplet of different concentrations of BSA solution and food dye solution as compared to similar parameters for a 2 μl droplet of water. All concentrations of BSA have similar evaporation rates and relatively similar dimensions as water. It is noted from FIGS. 9A-9F that the diameter is similar to water for all concentrations of BSA, and the height of all BSA droplets is consistently lower than that of water. Low concentrations of BSA have similar CL, but higher concentrations have higher CL due to the presence of molecules interacting with the surface. CA values remained SH for BSA until pressures were large enough to pin the droplet to the surface (˜250 Pa). No visual residue was observed on the surface because BSA cannot be observed by eye, but particles are confined to the reduced contact area after evaporation is complete. In various implementations, as a result of evaporation, the volume reduced up to 100× for low concentrations of BSA, and the contact area decreased by 3.5×. Since enhanced detection is needed for low concentrations (high concentrations can be detected by current techniques), enhancement of lower concentrations would have a high impact on diagnostics.

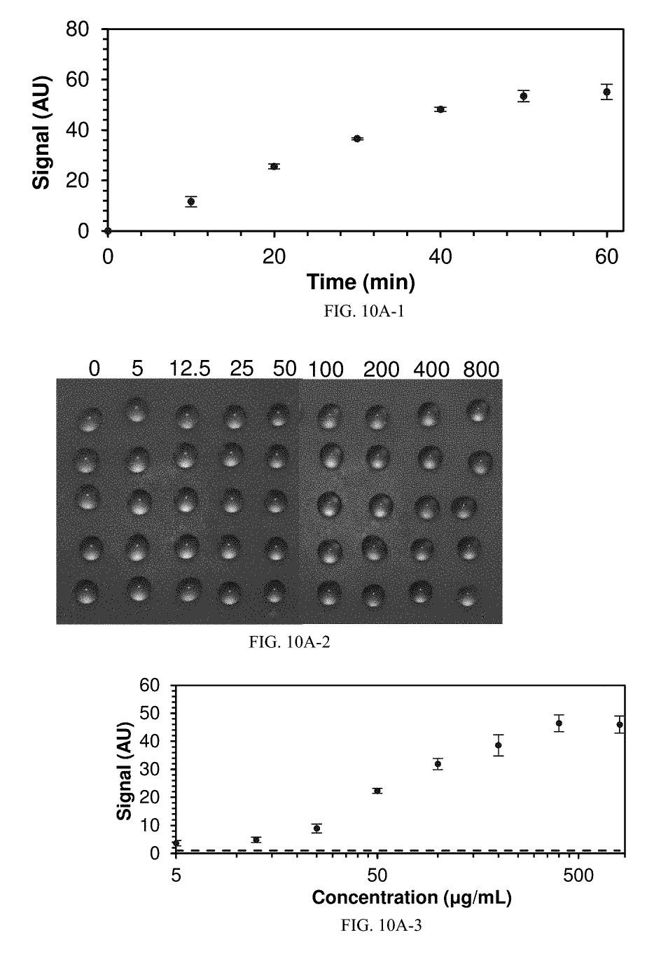

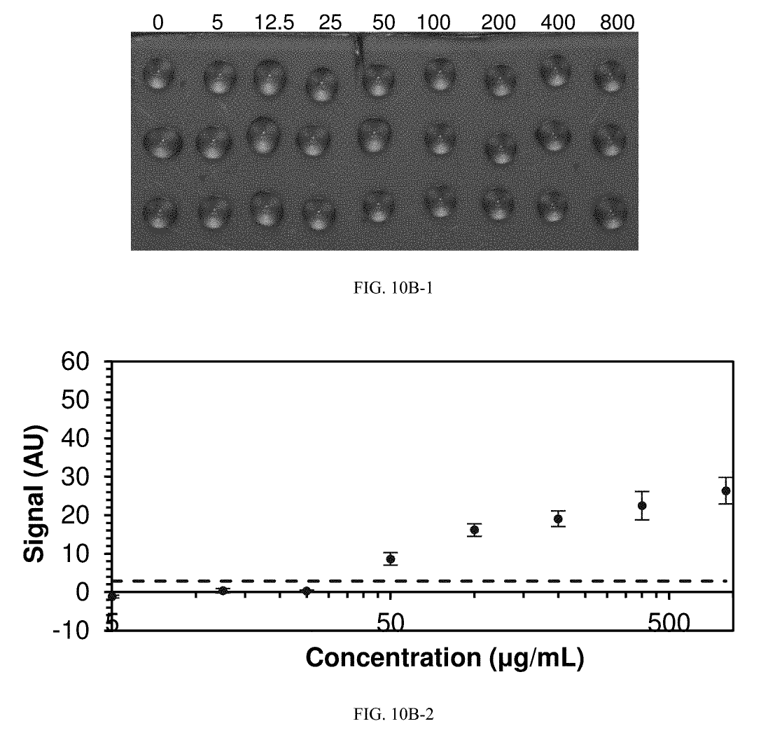

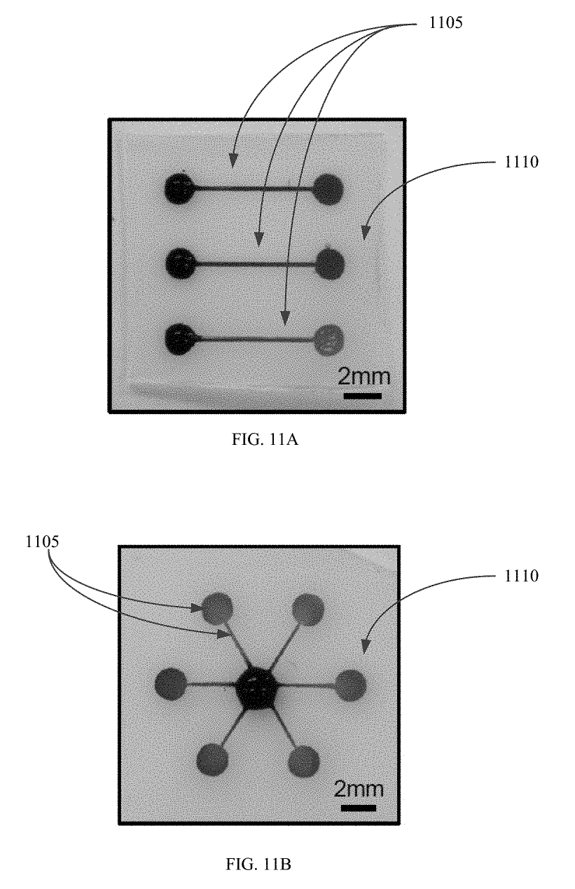

BSA was detected on the SH surface with detection dye, and the colorimetric signal was measured. Detection dye was added to the SH surface and evaporated for 60 minutes to allow concentrating. FIG. 10A-1 shows the increase in the colorimetric signal intensity over time of the detection dye resulting from evaporation on a SH surface. BSA was then added to interact with detection dye and change from brown to blue. The blue colorimetric signal was measured and compared to colorimetric signal obtained when BSA is added to a dye that was allowed to evaporate on a flat surface. FIG. 10A-2 illustrates the image of droplets of detection dye disposed on a SH surface mixed with different concentrations of BSA ranging from 0 μg/ml to 800 mg/ml.

FIG. 10A-3 shows the colorimetric signal intensity for different concentrations of BSA when mixed with detection dye disposed on a SH surface. From FIG. 10A-3, it is noted that BSA concentration as low as 5 μg/ml can be detected with this method. FIG. 10B-1 illustrates the image of droplets of detection dye disposed on a flat surface (or non-SH surface) mixed with different concentrations of BSA ranging from 0 μg/ml to 800 mg/ml. FIG. 10B-2 shows the colorimetric signal intensity for different concentrations of BSA when mixed with detection dye disposed on a non-SH surface. A comparison of FIGS. 10B-2 and 10A-3 indicates that when BSA is mixed with detection dye place on a flat surface, BSA concentration as low as 50 μg/mL can be detected. Moreover, the signal intensity is not as high as compared to the SH surface. Thus, BSA concentration cannot be accurately quantified based on signal intensity due to overlap in signal.

The evaporation, in addition to optical effects of the almost spherical droplet, improve the colorimetric detection signal, and a level of detection (LOD) lower than 10 μg/mL (e.g., 5 μg/mL) can be achieved in certain implementations. The signal intensity is distinguishable for all BSA concentrations tested, and therefore, BSA concentration can be quantified from signal intensity. Based on the curve in FIG. 10A-3, the calculated LOD is 1.3 μg/mL and concentrations up to 400 μg/mL are distinguishable on the SH surface. Concentrations greater than 400 μg/mL are detectable but not quantifiable

Evaporation on a SH surface concentrates molecules up to 402× and further reduces the contact area up to 4.75×. This concentrating effect leads to enhanced detection, and by evaporating on a SH surface, BSA can be detected at concentrations 10× lower than on a flat surface. The detection signal intensity on a SH surface is also greater than on a flat surface, and concentrations are distinguishable and can be quantified. This technique is simple to implement, is relatively fast (<1 hr), and does not require external processing or preparation. The colorimetric signal negates using expensive external equipment for detection, but this technique has could be integrated with more advanced detection techniques. In addition, the SH surfaces are simple and inexpensive to manufacture, making the technique affordable for low-cost diagnostics.

### Diagnostic Systems and Platforms

Diagnostic Systems including a detection zone comprising a SH surface can be advantageous in increasing the detection sensitivity of one or more chemical components in biological fluid. For example, in various implementations, a diagnostic system can comprise a platform including a detection zone for receiving a volume of biological fluid. The detection zone can have an area that is between about 10 μm² to about 1000 μm². The detection zone can include a plurality of micro-scale and/or nano-scale features that render the detection zone superhydrophobic. The SH detection zone can be fabricated by the fabrication method described in FIG. 2 or any other methods described herein or known to a person skilled in the art. For example, in various implementations, one or more detection zones can be fabricated by patterning one or more areas on a substrate and forming micro-scale and/or nano-scale features in the patterned area by the methods described herein.

The proteins and/or other molecules in the volume of biological fluid received on the detection zone can be concentrated by evaporating on the SH detection zone by the methods described above. A detector can be directed towards the detection zone to detect a property of the biological fluid and/or the nature and amount of the proteins and/or other molecules in the volume of biological fluid. In this manner, the diagnostic system can be adapted to detect and/or quantify an analyte (e.g., proteins and/or other molecules) in a volume of biological fluid. Due to the concentration effect, the diagnostic system can be adapted to detect and/or quantify an analyte (e.g., proteins and/or other molecules) even when present in low concentrations in the biological fluid. For example, in one implementation, bovine serum albumin (BSA) can be detected even when present in concentrations as low as about 5 μg/ml. Generally depending on the nature of the analyte, it is possible to detect analytes in biological fluids in concentrations as low as 0.1 μg/ml. For example, depending on the analyte, it is possible to detect analytes in biological fluids in concentrations as low as 1 μg/ml, as low as 2 μg/ml, as low as 3 μg/ml, as low as 4 μg/ml, as low as 5 μg/ml, as low as 10 μg/ml using a diagnostic system as disclosed herein. Since, the diagnostic systems described herein can be manufactured in a cost effective manner, the can advantageously increase the limit of detection in a cost effective manner. By virtue of their simplicity, inexpensive materials, ease of manufacturing and high sensitivity, the diagnostic systems described herein can be used in detecting and/or diagnosing many medical conditions including but not limited to the onset of pre-clampsia in pregnant women.

In various implementations, the diagnostic system can include microfluidic channels that can convey the volume of biological fluid towards the detection zone. The volume of biological fluids can be driven through the microfluidic channels using known methods such as a pressure difference or an electric potential difference. In various implementations, the microfluidic channels can be similar to the traditional microfluidic channels known to a person skilled in the arts. In various implementations, the microfluidic channels can be closed microfluidic channels that are adapted to be hydrophobic or super hydrophobic by providing a plurality of micro-scale and/or nano-scale features within the channels. The hydrophobic microfluidic channels can be fabricated by using methods described herein. In such implementations, the volume of biological fluid can be pressure driven or electrostatically driven through the hydrophobic or super hydrophobic channels. In such implementations, the volume of biological fluid can be driven through the microfluidic channels with reduced stiction.

In various implementations, the microfluidic channels can be open microfluidic channels that are adapted to be superhydrophilic. In such implementations, the region surrounding the superhydrophilic channel can be made hydrophobic or superhydrophobic by patterning the surrounding region with micro-scale and/or nano-scale features. In such implementations, the volume of biological fluid can be self-driven through the superhydrophilic microfluidic channels by using a difference in the hydrophobicity between the channel and its surrounding. Implementations of self-driven microfluidic channels are discussed in detail below.

### Self-Driven Microfluidic Channels

In various implementations, the diagnostic system can include self-driven microfluidic channels. Self-driven microfluidic channels include hydrophilic channels surrounded by a hydrophobic region such that a volume of biological fluid is self-driven through the microfluidic channel due to a difference in hydrophobicity between the channels and its surrounding.

FIGS. 11A and 11B show different implementations of a hydrophilic microfluidic channel 1105 surrounded by a hydrophobic region 1110. In various implementations, the hydrophobic region 1110 can include a plurality of micro-scale and/or nano-scale features as shown in FIGS. 3A and 3B. In various implementations, the hydrophobic region can be configured as a superhydrophobic region wherein a droplet of water has a contact angle greater than about 150 degrees and a sliding angle less than 25 degrees. The hydrophilic microfluidic channels can be patterned in a variety of shapes. For example, the hydrophilic microfluidic channels can be patterned as linear open channels and/or circular areas, as shown in FIG. 11A. In various implementations, the hydrophilic microfluidic channels can include a plurality of curvilinear segments.

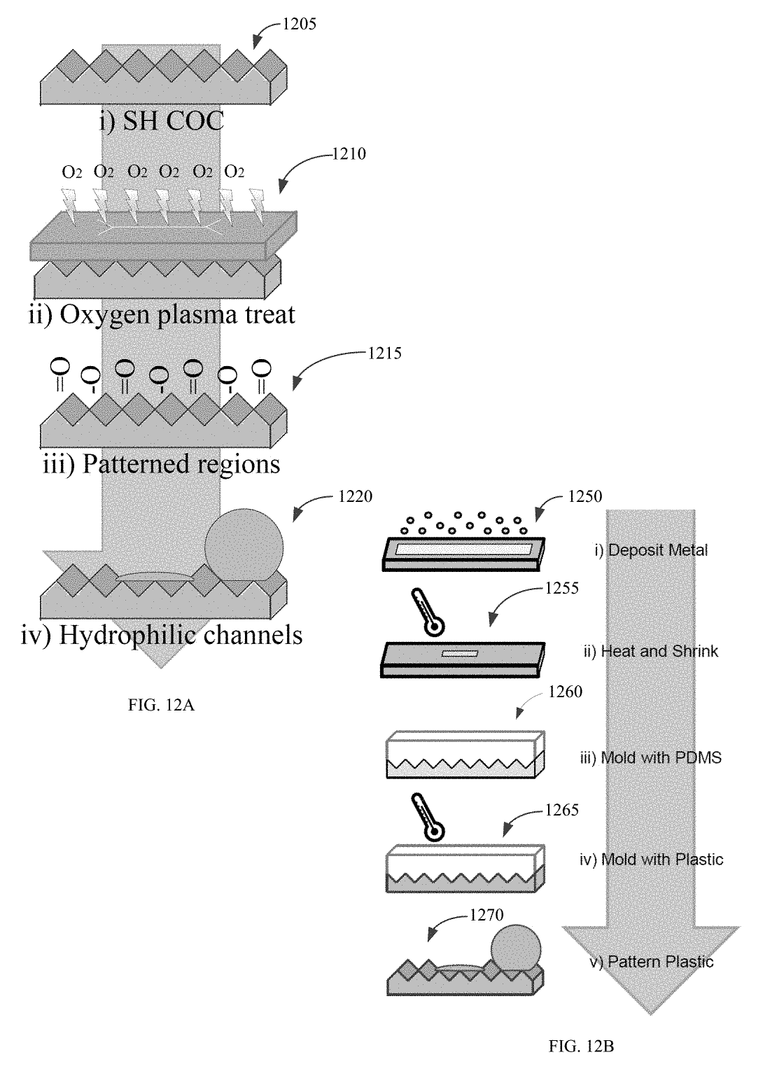

FIGS. 12A and 12B show different implementations of methods to fabricate hydrophilic microfluidic channels on a hydrophobic surface. The first implementation of a method to fabricate hydrophilic microfluidic channels on a hydrophobic surface includes providing a SH polymer substrate including micro-scale and nano-scale features as shown in block 1205. In the illustrated implementation, the SH polymer substrate can be a SH cyclo olefin copolymer (COC). The SH polymer substrate can be manufactured by a method described above in connection with FIG. 2. A negative mask is adhered to the SH polymer substrate as shown in block 1210. The negative mask includes patterns that exposes regions of the SH polymer substrate that are desired to be hydrophilic and masks regions of the SH polymer that are desired to be hydrophobic. The masked SH polymer substrate is oxygen plasma treated to render the exposed regions of the SH polymer substrate hydrophilic. Oxygen functional groups attach to the exposed surfaces of the SH polymer substrate during plasma treatment and render them more hydrophilic such that fluid disposed on the exposed surfaces can wet the exposed surfaces. The non-exposed surfaces remain SH.

The second implementation of a method to fabricate hydrophilic microfluidic channels on a hydrophobic surface includes depositing metal on a shrink film polymer (e.g., polyolefin) as shown in block 1250 of FIG. 12B. The metal coated shrink film polymer is heated as shown in block 1255 to create micro-scale and nano-scale features. The micro-scale and nano-scale features are molded with PDMS as shown in block 1260 and then transferred onto a plastic material (e.g., a hard plastic), for example, by hot embossing as shown in block 1265. Microfluidic channels are patterned on the SH plastic substrate and treated to render them hydrophilic.

In various implementations, the hydrophilic microfluidic channels can be incorporated with biomarkers (e.g., biotin, IgG, biotin-streptavidin, fluorescein, etc.) such that one or more analytes present in the biological fluid can be detected as the fluid flows through the microfluidic channel.

Diagnostic systems including microfluidic channels (either self-driven, pressure driven or electrostatically driven) can be integrated with platforms including SH detection zones with micro-scale and nano-scale features to enhance the detection sensitivity of analytes in biological fluids. Such diagnostic systems can be useful in inexpensive point-of-care (POC) devices that bridge the gap between patients and medical testing and allow diseases to be diagnosed with relatively quickly and inexpensively.

Several advantages of the systems and embodiments described herein are discussed herein above. Further, bodily fluid are also compatible with the SH surfaces fabricated in the manner discussed above such that blood, saliva, urine and other bodily fluids can be used as the testing fluid on the SH surfaces. SH surfaces fabricated in the manner discussed above have also been shown to prevent blood clotting, and the proposed surfaces can be used as an anticoagulation surface.

Other methods of fabricating SH surfaces include surface with structural and chemical modifications. The chemical modifications often make the surfaces not compatible for biological application. However, bodily fluids are compatible with the proposed SH surfaces because superhydrophobicity is created only by structural modifications which are then transferred into biocompatible materials.

Another advantage of the embodiments described herein is that self-driven microfluidic channels do not require external equipment, tubing, valving, and loss of reagents. They are also more easily fabricated compared to traditional microfluidics and can be used for broad applications and settings.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. A method of manufacturing a diagnostic device, the method comprising:
providing a superhydrophobic (SH) polymer substrate;
disposing a mask over the SH polymer substrate, the mask including a pattern that exposes a region of the SH polymer substrate and masks another region of the SH polymer substrate; and
treating the masked SH polymer substrate with oxygen plasma to render the exposed region hydrophilic, the masked region of the SH substrate remaining hydrophobic.

2. The method of manufacturing the diagnostic device of claim 1, wherein providing a superhydrophobic substrate comprises:
depositing a layer of metal on a polymer material;
shrinking the polymer material;
obtaining a mold by molding the metal coated shrunk polymer material onto a first moldable polymer;
molding a second moldable polymer material using the obtained mold.

3. The method of manufacturing the diagnostic device of claim 2, wherein the metal comprises at least one of silver, gold or calcium.

4. The method of manufacturing the diagnostic device of claim 2, wherein the polymer material comprises prestressed thermoplastic polyolefin (PO) or a shrink film PO.

5. The method of manufacturing the diagnostic device of claim 2, wherein the first or the second moldable polymer material comprises polydimethylsiloxane (PDMS).
